(19) United States
(12) Patent Application Publication
(10) Pub. No.: US 2025/0046473 A1
(43) Pub. Date: Feb. 6, 2025

(54) An Automated AI-Based Method And System For Creating And Providing Medical Guidelines

(71) Applicant: SERENUS AI LTD., Tel-Aviv (IL)

(72) Inventors: Josef Elidan, Tel-Aviv (IL); Orly Elidan-Harel, Hod-HaSharon (IL)

(21) Appl. No.: 18/685,253

(22) PCT Filed: Aug. 18, 2022
(86) PCT No.: PCT/IB2022/057749

§ 371 Date: Feb. 21, 2024

Related U.S. Application Data
(60) Provisional application No. 63/235,815, filed on Aug. 23, 2021.

(51) Int. Cl. G16H 70/20

(52) U.S. Cl. CPC G16H 70/20

(57) ABSTRACT

An automated AI-based system for creating and providing medical guidelines, comprising: a system server configured to communicate with at least one medical source; store medical data from the at least one medical source in a database; analyze at least a first part of the medical data; and store experts' labeling of at least a second part of the medical data; the system server comprises a machine learning module configured to create medical guidelines based on the at least first part of the medical data and the labeling.

### FIELD OF THE INVENTION

The present invention generally relates to the medical field and specifically to an automated AI-based method and system for creating and providing medical guidelines.

### BACKGROUND

Millions of treatments are provided each year worldwide. According to recent research, a high percentage of medical treatments/procedures are not appropriate, risking patients' lives, leading to poor outcomes and wasting valuable resources.

Today, medical guidelines are created manually in a process which depends on professional committees requiring valuable resources.

Moreover, the process of creating and publishing those medical guidelines is manual and may last a long time.

However, evidence-based research is constantly being published and updated leading to changes in the medical guidelines. In addition, there is no method in place to learn from the ongoing accumulated knowledge of medical professionals.

Therefore, there is an urgent need for a method and system for creating and updating medical guidelines to be updated and published continuously, improving patient outcomes and prognosis, saving lives and valuable resources.

### SUMMARY

According to an aspect of the present invention there is provided an automated AI-based system for creating and providing medical guidelines, comprising: a system server configured to: communicate with at least one medical source; store medical data from the at least one medical source in a database; analyze at least a first part of the medical data; and store experts' labeling of at least a second part of the medical data; the system server comprises a machine learning module configured to create medical guidelines based on the at least first part of the medical data and the labeling.

The at least one medical source may comprise at least one of internal data source and external data source.

The analysis of the at least first part of the medical data may be performed using at least one of Natural Language Processing (NLP) and artificial intelligence tools.

The at least one medical source may comprise at least one report generated by a reports and statistics module; and wherein the machine learning module may further be configured to create medical guidelines based on the at least first part of the medical data, the at least one report and the labeling.

The at least one medical source may comprise at least one synthetic medical case generated by the system; and wherein the machine learning module may further be configured to create medical guidelines based on the at least first part of the medical data, the at least one synthetic medical case and the labeling.

The at least one medical source may comprise prospective medical data; and wherein the machine learning module may further be configured to create medical guidelines based on the at least first part of the medical data, the prospective medical data and the labeling.

The at least one medical source may comprise at least one patient's outcome; and wherein the machine learning module may further be configured to create medical guidelines based on the at least first part of the medical data, the at least one patient's outcome and the labeling.

According to another aspect of the present invention there is provided an automated AI-based method of creating and providing medical guidelines, comprising: retrieving medical data from at least one medical source and storing the retrieved data; analyzing at least a first part of the retrieved medical data; labeling at least a second part of the retrieved medical data; and using a machine learning module for creating medical guidelines based on the at least first part of the retrieved medical data and the labeling.

The at least one medical source may comprise at least one of internal data source and external data source.

The analysis of the at least first part of the medical data may be performed using at least one of Natural Language Processing (NLP) and artificial intelligence tools.

The at least one medical source may comprise at least one report generated by a reports and statistics module; and wherein the method may further comprise using the machine learning module for creating medical guidelines based on the at least first part of the medical data, the at least one report and the labeling.

The at least one medical source may comprise at least one synthetic medical case; and wherein the method may further comprise using the machine learning module for creating medical guidelines based on the at least first part of the medical data, the at least one synthetic medical case and the labeling.

The at least one medical source may comprise prospective medical data; and wherein the method may further comprise using the machine learning module for creating medical guidelines based on the at least first part of the medical data, the prospective medical data and the labeling.

The at least one medical source may comprise at least one patient's outcome; and wherein the method may further comprise using the machine learning module for creating medical guidelines based on the at least first part of the medical data, the at least one patient's outcome and the labeling.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 is a schematic block diagram of the system, according to embodiments of the present invention;

FIG. 2 is a schematic block diagram of an exemplary implementation of the structure of a single treatment entry in the system database;

FIG. 3 is a flowchart showing the steps taken during an exemplary session with a user for creating a medical record;

FIG. 4 is a flowchart showing the process performed by the system of present invention in order to predict a treatment's efficacy;

FIG. 5 is a flowchart showing an exemplary process of creating a synthetic or semi-synthetic file, according to embodiments of the present invention;

FIG. 6 is a flowchart showing an exemplary process, performed by the system of the present invention, after the generated files have minimal to no contradictions; and

FIG. 7 is a flowchart showing an exemplary process of creating medical guidelines, according to embodiments of the present invention.

### DETAILED DESCRIPTION OF PREFERRED EMBODIMENTS

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is applicable to other embodiments or of being practiced or carried out in various ways. Also, it is to be understood that the phraseology and terminology employed herein is for the purpose of description and should not be regarded as limiting.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire line, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The operations and algorithms described herein can be implemented as executable code within the control unit—and/or processor circuit—as described, or stored on a standalone computer or machine readable non-transitory tangible storage medium that are completed based on execution of the code by a processor circuit implemented using one or more integrated circuits. Example implementations of the disclosed circuits include hardware logic that is implemented in a logic array such as a programmable logic array (PLA), a field programmable gate array (FPGA), or by mask programming of integrated circuits such as an application-specific integrated circuit (ASIC). Any of these circuits also can be implemented using a software-based executable resource that is executed by a corresponding internal processor circuit such as a microprocessor circuit (not shown) and implemented using one or more integrated circuits, where execution of executable code stored in an internal memory circuit (e.g., within a memory circuit) causes the integrated circuit(s) implementing the processor circuit—to store application state variables in processor memory, creating an executable application resource (e.g., an application instance) that performs the operations of the circuit as described herein. Hence, use of the term “circuit” in this specification refers to both a hardware-based circuit implemented using one or more integrated circuits and that includes logic for performing the described operations, or a software-based circuit that includes a processor circuit (implemented using one or more integrated circuits), the processor circuit including a reserved portion of processor memory for storage of application state data and application variables that are modified by execution of the executable code by a processor circuit. The memory circuit can be implemented, for example, using a non-volatile memory such as a programmable read only memory (PROM) or an EPROM, and/or a volatile memory such as a DRAM, etc.

An automated AI-based method and system for creating and providing medical guidelines to medical treatments pathways is provided. It will be appreciated that the term ‘treatment’ may include, but is not limited to, any medical procedure or any medical treatment including but not limited to surgeries, medication, cancer management etc. The method and system of the present invention collects, maps and analyzes medical data, enables experts to label the data and provides medical guidelines accordingly using machine learning capabilities. The method and system of the present invention may use knowledge of most recent research in each field, statistics, experts' knowledge, experts' labeling of real and/or simulated cases, prospective data including objective indicators of patients' outcomes, patients' and professionals' feedback, as well as machine learning technologies.

The system database is typically constantly reviewed and updated, using research and machine learning techniques, based on one or more of: professionals and patients' feedback, physicians and supervisors' decisions (labels), objective patient outcomes indicators such as but not limited to: readmissions, physician visits records, complications, additional treatments provided, research and a large number of medical records, including external data received, for example, from hospitals, health maintenance organizations (HMOs) and the like. It will be appreciated that the terms ‘user’ and ‘patient’ may be used intermittently throughout the hereinbelow description.

The training of the system is developed from several sources:
Existing research and guidelines.
Experts' Knowledge and labeling.
Historical and/or simulated data (due to lack of sufficient historical data), such as synthetic cases as will be detailed below.
Real prospective data and treatment efficacy as will be detailed below.

Following a solid expert system and a learned improvement in place, the next step is to use real-world data to continuously improve the system using advanced learning machinery by allowing for a greater breadth of measurements, “big data” scale of data processing using advanced machine learning techniques such as random forests or deep learning. At the high level, at the context of the present invention, four axis of data and ML progression may be pursued in parallel:
1. User-Based Records. As noted above, one of the benefits of putting a reasonable baseline in place is that this model can be used to provide a useful (even if not perfect) service to users (patients). By simply using the system, each user creates a real record, which can then be labeled by an expert relatively quickly. Further, user follow-up feedback can serve as a (noisy) automated labeling mechanism which can further improve the system, similar to user labeling of images in a personal photo album. The obvious benefit of this axis is the low-cost accumulation of real cases.
2. Unlabeled Medical Records. Medical records from the practices of experts and/or from medical institutions can similarly be labeled by experts. This can allow learning techniques to be applied to much richer measurement spaces, taking into account a wealth of information that goes well beyond what can be captured by a hand-coded scenario. While ultimately of higher quality than (1), this axis of progress is naturally slower and should just be pursued in parallel. Importantly, error analysis of models learned as part of (1) can act as a guide for informed collection of records, speeding up this stage.
3. Labeled Medical Records. Many health and insurance institutions also have medical records that have been labeled by the treating doctor. The obvious benefit is the availability of a large number of labeled records. While the quality of the labels is likely to be lower than that of the best physician in the field, mixture of experts' machine learning techniques can be used to mitigate this problem (often surpassing the best expert). Ultimately this fully automated axis is likely to lead to the biggest progress, simply due to the high-volume of (reasonably) labeled examples.
4. Labeled Simulated Medical Records. Since some of the needed dataset does not exist in real medical records, the information can be completed by simulated data according to, for example, the real distribution of the population.

The system operates, but is not limited to operate, on a number of levels:
1. Providing a detailed report, including patient's responses to factors presented in a personalized chatbot or questionnaire, detailing the relative dynamic impact (positive or negative) of each factor.
2. Storing detailed anonymous medical information relating to patients.
3. Storing experts labeling of real medical records and/or simulated cases (“wisdom of the crowd”)
4. Acquiring large volumes of prospective information referring to patient outcomes to be processed by the system's machine learning modules.
5. Creating medical guidelines according to the accumulated medical knowledge.
6. Improving the accuracy of the system by using machine learning modules and by analyzing at least some of: a large number of patients' records (big data), simulated cases labeling, users' feedback (prospective data) and patients' outcomes.

The system uses, but not limited to use, a number of novel technologies including:
1. A dynamic algorithm for building a personalized set of factors responses with a dynamic weight for each factor and an accumulated weight to create a personalized chatbot to collect patients' anonymous data.
2. A machine learning training system that generates and calibrates simulated medical records (for training purposes, in order to overcome lack of sufficient data in historical data).
3. A machine learning module that determines and improves the weight/impact of each relevant factor and final recommendation for the treatment being considered according to experts labeling of real and/or simulated medical cases.
4. A machine learning module that interfaces with medical records and objective patient outcomes indicators such as and not limited to readmissions, complications etc. and professionals' and patients' feedbacks for the purpose of finding new indications and hidden correlations between data of a plurality of patients regarding the recommendations given for treatments, thereby predicting treatment efficacy, and improving the overall precision of the system.
5. A machine learning module that improves the medical guidelines over time.

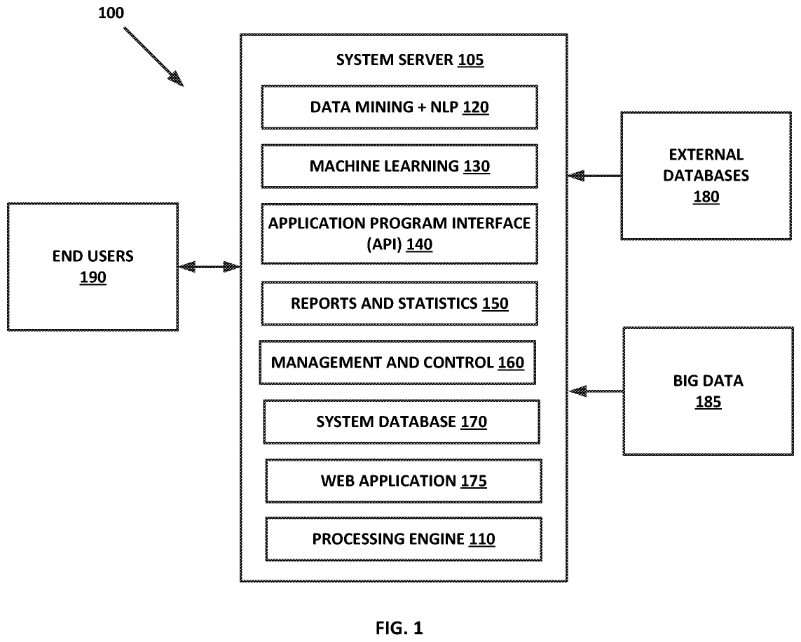

FIG. 1 is a schematic block diagram of the system, according to embodiments of the present invention.

System 100 comprises one or more system servers (only one shown) 105, preferably a web server, communicating over the Internet with external databases 180, such as medical institutions' databases comprising patients' files, with big data resources 185, including both structured and unstructured data and with end users' electronic communication means 190, such as medical institutions' systems and patients' computers and/or mobile electronic communication devices.

System server 105 comprises a processor and some or all of the following computerized modules:
A data mining and Natural Language Processing (NLP) module 120, configured to extract information from external databases 180 and transform it into an understandable structure for further use, using NLP techniques. Data extracted includes, for example, data from patients' medical files such as lab reports, free text notations etc. The extracted data is used for automatic labeling for training the machine learning module.
A machine learning module 130, configured to some or all of:
   Calibrate the weight (impact) of each variable relevant to each treatment and factor, by analyzing a large number of scenarios and their labels.
   Calibrate the system using information mined from prospective real medical files (Big Data) or simulated cases (synthetic cases).
   Calibrate the system using professionals and patients' feedback after having undergone the treatment.
   Calibrate the system by scanning latest researches, statistics and publications by health organizations (e.g. American Academy Guidelines, World Health Organization, American and European health organizations, etc.).
   Find connections and correlations between patients' profiles and patients' outcomes as results of treatments for predicting treatment efficacy using some or all of patients' data and reports received from at least one of the data mining and (NLP) module 120, the external databases 180, the big data resources 185 and the reports and statistics module 150.
   Create medical guidelines according to at least some of the above collected data, results, analysis and the like.
An Application Program Interface (API) module 140 configured to enable data retrieval from various external medical sources.
A reports and statistics module 150 configured to generate personal reports to patients following a factor and response session and to provide statistics calculated from a plurality of reports.
A management and control module 160 configured to manage the system including managing fields, treatments, factors and possible responses, databases, scores spectrums, impacts of responses, export and import information for machine learning purposes, managing clients, managing a combination of treatments. Managing-adding, editing and removing records. The management system is role and permissions based and is constantly being updated and evolved.
One or more database 170, storing:
   Patients' anonymous clinical information including lab test results, anamnesis and reports generated by the reports and statistics module 150;
   A set of specific queries and possible responses for each treatment, which are generated in advance, e.g., by human experts;
   A set of weights associated with each factor, which are pre-determined according to general knowledge and continuously and dynamically updated by the latest researches, statistics and guidelines of the American and European Academies, and by machine learning modules 130;
   Connections and correlations between patients' anonymous profiles as extracted from medical records or input by an interactive chatbot, treatment protocols and patients' outcomes as results of treatments for predicting treatment efficacy.
   Medical guidelines.
   Experts labeling of real and/or simulated medical records.
A web application 175, providing users with an interactive platform for communicating with the system over the Internet, including presenting queries, receiving responses, receiving reports and recommendations and a prediction to treatment efficacy.
A processing engine 110, configured to:
   Select and present one query at a time to the user;
   Dynamically grade user's responses according to currently associated weight, based on machine learning modules;
   Determine next query based on last response and the accumulated weight and optionally the weight of the previous response;
   Dynamically update weights according to previous and following responses and associated weights.
   Provide results to reports and statistics module 150.

Typically, in a set-up phase, a set of specific factors for each patient and treatment are generated in advance e.g. by human experts.

The factors are organized in a hierarchic flowchart in complex relations and each response receives a different weight (impact) according to a specific scenario, the previous factor and weight and the accumulated weight and treatment which, as said above, may be dynamically updated throughout the process, based on machine learning modules.

The hierarchical flow and weights (impacts) may be organized in any suitable logical relationship or structural combination. It is possible that each case shall receive a different set of factors and that different weights may be assigned to the same factors in different combinations, e.g., so as to yield a specific output for each individual end-user.

As a result and during the process, each user views a personally customized dynamic scenario, according to the selected assigned treatment, and the user's responses.

At the end of the process, the weights received or updated for all the responses are analyzed to provide the relevant output.

According to certain embodiments, a patient facing a particular treatment is asked relevant factors regarding his medical condition.

According to embodiment of the invention, some responses may be pulled automatically by the system from the patient's medical records using NLP techniques.

Each response receives a certain dynamic impact value, according to the relative importance and impact (i.e., weight) on the decision to conduct the specific treatment. If the response negates the treatment, it receives a negative impact.

At the end of the process the system analyzes the input and the patient and/or the medical specialists receives a result, with the relative indication or contraindication for the treatment and additional recommendations e.g., further tests and conservative treatments needed before undergoing the treatment.

Examples of results:
Low indication for the treatment.
Moderate indication for the treatment.
High indication for the treatment.
Equivocal results. A second opinion or further discussion is needed.
The treatment is not justified.
The process is terminated because some crucial information is missing.
The process is terminated because more evaluation (test) is needed.

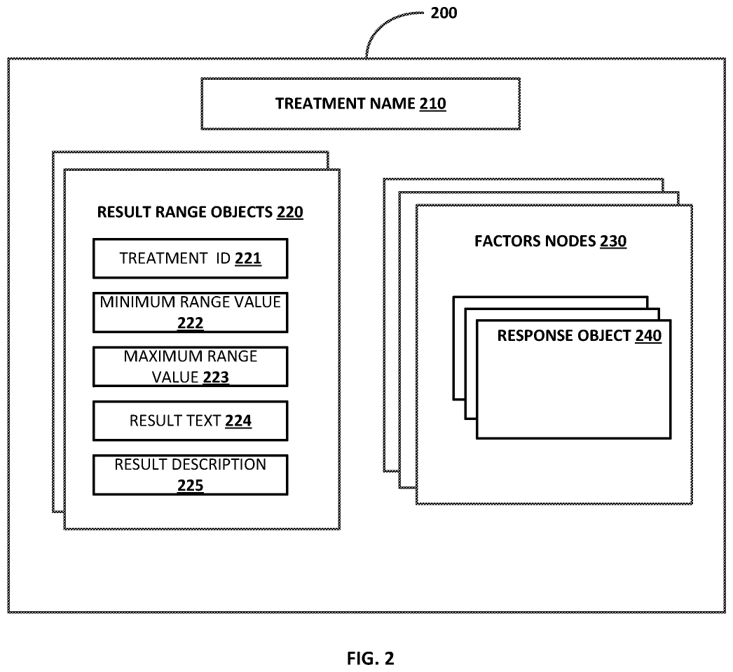

FIG. 2 is a schematic block diagram of an exemplary implementation of the structure of a single treatment entry 200 in the system database 170.

A treatment entry 200 may comprise some or all of the following objects:
Treatment name 210;
A set of result range objects (RRO) 220;
A multi-dimensional tree of factor nodes (FN) 230 and response object 240 for each factor node.

A Result Range Object (RRO) 220 may for example comprise some or all of the following objects:
Treatment ID 221;
Minimum Range Value (MINRV) (number) 222;
Maximum Range Value (MAXRV) (number) 223;
Result Text 224;
Result Description 225.

A factor node (FN) 230 may for example comprise some or all of the following objects:
Factor text (text);
Factor priority;
Required keys for unlocking the factor (optional);
Indication whether the factor has an automatic response;
Visual Analog Scale (VAS) range (min and max);
Min score to show factor;
Max score to show factor;
A set of response objects (RO).

A response object (RO) 240 may for example comprise some or all of the following objects:
Response text (text);
Response weight (integer-positive or negative);
Response key (AK) or any other indicator for automatically selecting the next factor node.
Automatic keys to fire response;
Force score to assign to user;
Indication of test end.

The keys serve for tracking selected ones of the user's responses and may change the course of the session by selecting the next factor, determining whether the session should terminate and force a score.

The keys method may be replaced by any other method for automatically identifying which factors are relevant to a particular context, given a collection of possible factors that are relevant to a treatment, the factors dynamic weight and the accumulated weight.

Three components, together or independently, contribute to these methods:
1) An expert prior indicating the relevancy of a particular factor given a particular response to another factor.
2) An expert label that is generated as follows: factors are presented to the expert, either one after the other or as a complete scenario and the expert evaluates the relevancy of each factor to the given context (either as a numerical score or a categorical one, e.g., “relevant”, “not relevant”).
3) Factors are identified as irrelevant based on statistical evaluation of relevancy to the target diagnosis task from past records.

The learning system may use either one or all of these components to learn a unified prediction system for the relevancy of a factor in a particular context. This relevancy “score” will be used to automatically modify the structure of the scenario. Another method that may replace keys:

Factor tree-A data structure that comprises nodes or factors and responses nodes in which each factor is a node and has a parent response.

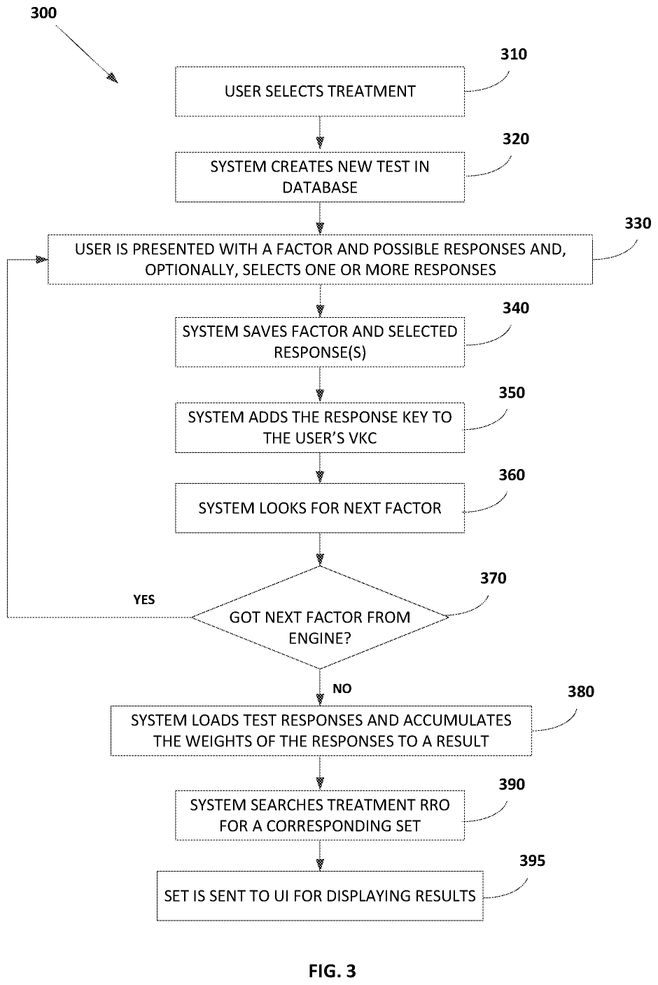

FIG. 3 is a flowchart 300 showing the steps taken during an exemplary session with a user for creating a medical record.

In step 310, the user selects a treatment from a given list of treatments and clicks “start test”.

In step 320, a new test is created in the system database 170, which may be a proprietary data repository. The test may include a timestamp, user info (IP etc.). A unique test ID is created.

During the test, the user typically sees the factors, ordered by their priorities. In step 330, the user is presented with a factor and, optionally, one or more possible responses to select from and selects the appropriate one or more responses.

The system then performs one or more of the following:
Saves the factor and selected response to a test database, with connection to the test ID (step 340).
Adds the response key (AK) (if exists) to the user's virtual key-chain (VKC) which comprises all current test response keys (step 350).

In step 360, the system checks in system's treatment's factors database for the next factor that matches some or all of the following criteria:
A factor with lower priority.
A factor that can be unlocked using the user's current VKC (looping through all existing keys and matching with factors' required keys).

In step 370, if the system found a new factor, it returns the new factor object to the user interface and loops back to step 330.

If no new factor was found, the system loads all the user's test responses from the database and accumulates the weight of the responses to a result (step 380).

It will be appreciated that the term ‘accumulates’ may include any mathematical operation or equation and the present invention is not limited to a simple sum operation.

In step 390, the system searches the treatment's RRO for the corresponding set, where result is between MINRV and MAXRV, and sends the found set to the user interface for results display (step 395).

At the end of this process a new medical record is created.

It is appreciated that the hierarchical flow and weights/points may be organized in any suitable logical relationship or structural combination. It is possible that different sets of factors and responses appear to each user and that different weights may be assigned for the same responses in different combinations, e.g., so as to yield a specific output for each individual end-user.

For some treatments, certain responses provided by an end-user may be deemed an absolute contraindication for the specific treatment and/or to the general/local anesthesia which are needed to conduct the treatment that was selected by the end-user, or may optionally result in the server presenting a recommendation for further information or examinations, another mode of treatment (e.g., conservative treatment) or another treatment entirely.

The tool can be presented in any digital platform and may provide end users with information on some or all of: treatments, descriptions, risks, statistics, tables, diagrams and drawings, along with automated decision making as described herein, thereby to enhance patients' ability to make more cautious medical decisions based on maximum information.

According to embodiments of the present invention, as mentioned above, the system may predict treatment efficacy, thereby improving the creation of the medical guidelines.

In order to find connections and/or correlations between patients' profiles, treatment protocols and patients' outcomes as results of treatments for predicting treatment efficacy, the machine learning module 130 collects and processes treatment protocols, patients' outcomes and patients' profiles including patients' data and reports related to various treatments, received from at least one of the data mining and (NLP) module 120, the external databases 180, the big data resources 185 and the database 170 storing reports generated by the reports and statistics module 150.

The patient's profile includes, but it not limited to include, patient's anonymous clinical history indicating treatment outcomes such as readmissions, complications, revisits, patients' and professionals' feedback and more.

The machine learning module 130 analyzes the collected data and finds correlations between patients' profiles, treatment protocols and patients' outcomes due to treatments, thereby enabling to predict treatment efficacy.

The analysis performed by the machine learning module 130 is constantly being updated and evolved, thereby enabling to provide more accurate results over time.

Hence, the system of the present invention enables to predict a desired treatment efficacy thereby improving the creation of the medical guidelines.

With a solid expert system and a learned improvement in place, the system of the present invention uses real-world data to continuously improve the process using advanced learning machinery by allowing a greater breadth of measurements, “big data” scale of data processing using advanced machine learning techniques such as, for example, random forests or deep learning.

Emphasis shall be on collecting objective indicators with regards to patient outcomes, such as, but not limited to, readmissions, complications, visits, patients' feedbacks and professionals' feedbacks.

Methods of finding insights between the data collected from historical data or the personalized chatbot, treatment protocols and the patient outcomes are implemented.

It will be appreciated that the system of the present invention is not limited to using the reports generated by the present invention. Accordingly, any medical data related to the patient, may be used.

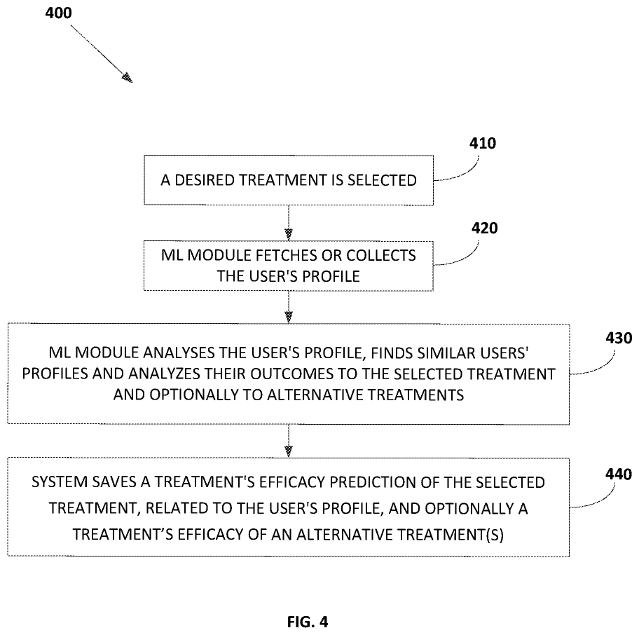

FIG. 4 is a flowchart 400 showing the process performed by the system of present invention in order to predict a treatment's efficacy.

In step 410, a desired treatment is selected.

In step 420, the ML module fetches (if available) or collects the user's profile.

The user's profile may include the user's data and reports related to various treatments the user has undergone in the past, received from at least one of the data mining and (NLP) module 120, the external databases 180, the big data resources 185 and the database 170 storing reports generated by the reports and statistics module 150.

In step 430, the ML module analyzes the user's profile, finds similar users' profiles and analyzes their outcomes to the selected treatment and optionally to alternative treatments.

In step 440, the system saves a treatment's efficacy prediction of the selected treatment, related to the user's profile, and optionally a treatment's efficacy of an alternative treatment(s).

According to embodiments of the present invention, as mentioned above, the system may create synthetic and/or semi-synthetic cases database, thereby improving the creation of the medical guidelines. The system and method of the present invention may create a large number of diverse synthetic and/or semi-synthetic medical files which may be improved over time.

It will be appreciated that throughout the specification the synthetic and/or semi-synthetic files may be referred to as cases or scenarios.

At the first stage, by marking conflicts between features within the synthetic and/or semi-synthetic files and setting probability of their occurrence, the system may learn how to create optimized cases in the future, according to the specific issue (e.g., disease).

At the second stage, by labeling the cases, the system is able to learn how to better calculate the defined variables, and improve the obtained conclusions throughout time.

The system creates diverse files which may include real data and synthetic data. The creation of the synthetic and/or semi-synthetic files is made based on labeling generated cases, with marking conflicts.

According to embodiments of the present invention, after a synthetic and/or semi-synthetic file is generated, it is presented to an expert (e.g., physician). The expert examines the file's data, validates that the parameters are relevant to the medical case and that there are no contradictions between the parameters.

According to embodiments of the present invention, the expert (physician) may mark the probability of different parameters to be presented in a file (percentage).

Every file inspected by the expert is saved by the system in order to enable the system to learn and create improved and more realistic files in the future.

It will be appreciated that the system of the present invention is not limited to saving all the files.

According to embodiments of the present invention, when the system generates a file with minimal to no contradictions, an expert (e.g., physician, the same or different from the first physician) may decide whether the file justifies a procedure (e.g., a medical operation) by labeling the case.

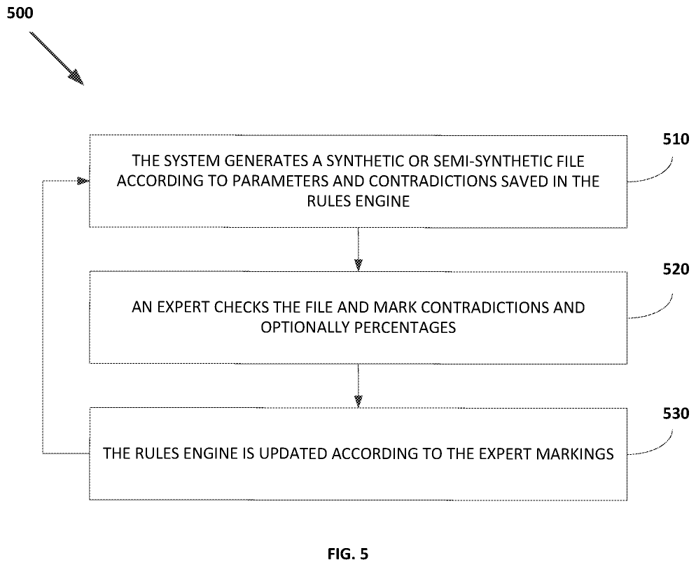

FIG. 5 is a flowchart 500 showing an exemplary process of creating a synthetic or semi-synthetic file, according to embodiments of the present invention.

In step 510, the system generates a synthetic or semi-synthetic file according to parameters, rules and contradictions saved in the rules engine.

In step 520, an expert checks the file and marks full contradictions between the answers (0% chance of future appearance) or optionally percentages.

In step 530, the rules engine is updated according to the expert's markings.

The process then may return to step 510 up to a point where the generated files have minimal to no contradictions.

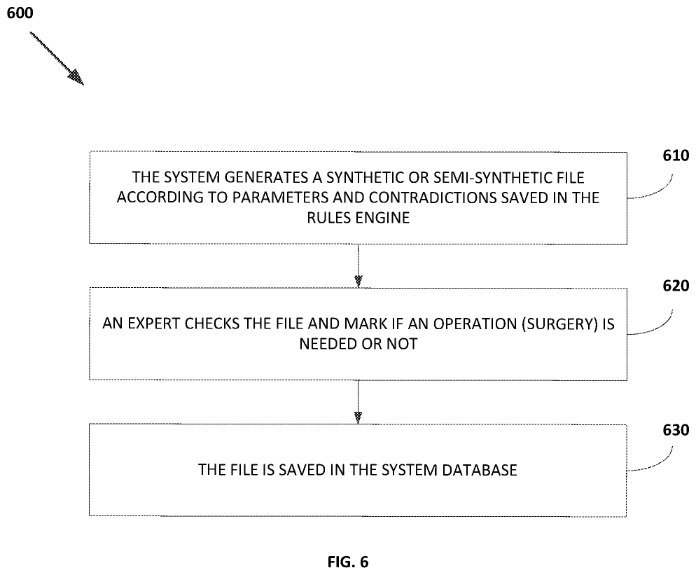

FIG. 6 is a flowchart 600 showing an exemplary process performed by the system of the present invention after the generated files have minimal to no contradictions.

In step 610, the system generates a synthetic or semi-synthetic file according to parameters, rules and contradictions saved in the rules engine.

In step 620, an expert checks the file and mark if a medical procedure is indicated (appropriate), not indicated (not appropriate), or the case needs further consideration (equivocal).

In step 630, the file is saved in the system database.

As mentioned above, in a set-up phase, a set of specific parameters, rules and contradictions for each medical/surgical procedure are generated, e.g., by human experts, in advance and saved in the rules engine.

Furthermore, these parameters, rules and contradictions may be updated according to updates in the research, statistics and new guidelines.

As a result of the processes described in conjunction with FIG. 5 and FIG. 6, the system may automatically generate a vast number of random synthetic and/or semi-synthetic files.

Using at least some of the above described data, the system of the present invention may create medical guidelines.

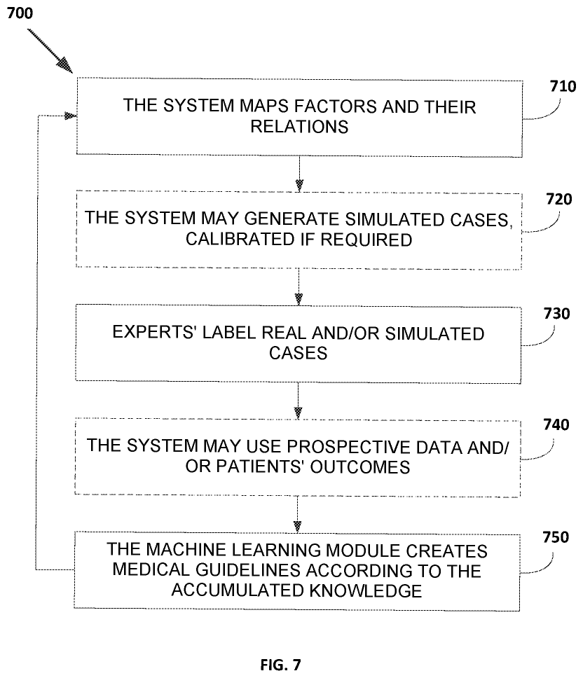

FIG. 7 is a flowchart 700 showing an exemplary process of creating medical guidelines, according to embodiments of the present invention.

The guidelines are created by the following:
1. Correlations found by labeled real and/or simulated medical records labeled by medical experts. For example, finding high fever as a factor constantly labeled as leading to indication for a procedure. Then creating a medical guideline, marking high fever as a factor to indicate a positive indication for a procedure.
2. Correlations found by prospective learning revealing hidden correlations between patients' anonymous profiles, treatment protocols and patients' outcomes. For example, finding correlation between not implementing sufficient physiotherapy before, for example, an Orthopedics surgery. Thereafter, creating a medical guideline marking sufficient physiotherapy before an Orthopedics surgery referral.

In step 710, the system maps factors and their relations according to at least some of: current guidelines, research, existing medical records, experts' knowledge, reports generated by the present invention or any other input known from the current wisdom of medicine.

If there is missing data, in step 720, the system may generate simulated cases, calibrated if required, according to, for example, the real distribution of the population.

In step 730, experts' label real and/or simulated cases.

In step 740, the system may use prospective data and/or patients' outcomes.

In step 750, the machine learning module creates medical guidelines according to the accumulated medical knowledge.

The process returns to step 710 and therefore, the system continuously improves the medical guidelines over time.

Features of the present invention, including method steps, which are described in the context of separate embodiments, may also be provided in combination in a single embodiment. Conversely, features of the invention, which are described for brevity in the context of a single embodiment or in a certain order may be provided separately, or in any suitable sub-combination or in a different order.

Any or all of computerized output devices or displays, processors, data storage and networks may be used as appropriate to implement any of the methods and apparatus shown and described herein.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

1. An automated AI-based system for creating and providing medical guidelines, comprising:
a system server configured to:
communicate with at least one medical source;
store medical data from said at least one medical source in a database;
analyze at least a first part of said medical data; and
store experts' labeling of at least a second part of said medical data;
said system server comprises a machine learning module configured to create medical guidelines based on said at least first part of said medical data and said labeling.

2. The system of claim 1, wherein said at least one medical source comprises at least one of internal data source and external data source.

3. The system of claim 1, wherein said analysis of said at least first part of said medical data is performed using at least one of Natural Language Processing (NLP) and artificial intelligence tools.

4. The system of claim 1, wherein said at least one medical source comprises at least one report generated by a reports and statistics module; and wherein said machine learning module is further configured to create medical guidelines based on said at least first part of said medical data, said at least one report and said labeling.

5. The system of claim 1, wherein said at least one medical source comprises at least one synthetic medical case generated by said system; and wherein said machine learning module is further configured to create medical guidelines based on said at least first part of said medical data, said at least one synthetic medical case and said labeling.

6. The system of claim 1, wherein said at least one medical source comprises prospective medical data; and wherein said machine learning module is further configured to create medical guidelines based on said at least first part of said medical data, said prospective medical data and said labeling.

7. The system of claim 1, wherein said at least one medical source comprises at least one patient's outcome; and wherein said machine learning module is further configured to create medical guidelines based on said at least first part of said medical data, said at least one patient's outcome and said labeling.

8. An automated AI-based method of creating and providing medical guidelines, comprising:
retrieving medical data from at least one medical source and storing said retrieved data;
analyzing at least a first part of said retrieved medical data;
labeling at least a second part of said retrieved medical data; and
using a machine learning module for creating medical guidelines based on said at least first part of said retrieved medical data and said labeling.

9. The method of claim 8, wherein said at least one medical source comprises at least one of internal data source and external data source.

10. The method of claim 8, wherein said analysis of said at least first part of said medical data is performed using at least one of Natural Language Processing (NLP) and artificial intelligence tools.

11. The method of claim 8, wherein said at least one medical source comprises at least one report generated by a reports and statistics module; and wherein said method further comprises using said machine learning module for creating medical guidelines based on said at least first part of said medical data, said at least one report and said labeling.

12. The method of claim 8, wherein said at least one medical source comprises at least one synthetic medical case; and wherein said method further comprises using said machine learning module for creating medical guidelines based on said at least first part of said medical data, said at least one synthetic medical case and said labeling.

13. The method of claim 8, wherein said at least one medical source comprises prospective medical data; and wherein said method further comprises using said machine learning module for creating medical guidelines based on said at least first part of said medical data, said prospective medical data and said labeling.

14. The method of claim 8, wherein said at least one medical source comprises at least one patient's outcome; and wherein said method further comprises using said machine learning module for creating medical guidelines based on said at least first part of said medical data, said at least one patient's outcome and said labeling.
